(19) United States
(12) Patent Application Publication
(10) Pub. No.: US 2025/0258179 A1
(43) Pub. Date: Aug. 14, 2025

(54) Method for Estimating Meal Component Bioavailability

(71) Applicants: Nicolaas Deutz, College Station, TX (US); Marielle Engelen, 1405 Mission Hills Ct., TX (US); Savanah Knezek, College Station, TX (US)

(72) Inventors: Nicolaas Deutz, College Station, TX (US); Marielle Engelen, 1405 Mission Hills Ct., TX (US); Savanah Knezek, College Station, TX (US)

(73) Assignee: The Texas A&M University System, College Station, TX (US)

(21) Appl. No.: 19/049,324

(22) Filed: Feb. 10, 2025

Related U.S. Application Data
(60) Provisional application No. 63/551,601, filed on Feb. 9, 2024.

(51) Int. Cl. G01N 33/68

(52) U.S. Cl. CPC G01N 33/6803; G01N 2458/15; G01N 2800/02

(57) ABSTRACT

Provided herein are methods for measuring bioavailability of nutrients in a subject after a meal, modeling nutrient metabolism in a subject in need thereof and monitoring nutrient requirements in a patient. The methods utilize a combination of stable isotope-labeled pulse tracers and compartmental analysis to determine at least bioavailability of amino acids in the meal or intracellular amino acid responses to meal components. Also provided is a profile of nutrient metabolism produced by the methods.

### CROSS-REFERENCE TO RELATED APPLICATIONS

This non-provisional patent application claims benefit of priority under 35 U.S.C. § 119 (e) of provisional patent application U.S. Ser. No. 63/551,601, filed Feb. 9, 2024, the entirety of which is hereby incorporated by reference.

### STATEMENT REGARDING FEDERALLY SPONSORED RESEARCH

This invention was made with government support under Grant Number HL132887 awarded by the National Institutes of Health from the National Heart, Lung, and Blood Institute. The government has certain rights in the invention.

### BACKGROUND OF THE INVENTION

### Field of the Invention

The present invention relates generally to the field of nutrient metabolism. More specifically, the present invention relates to methods for estimating meal component bioavailability.

### Description of the Related Art

Although it is generally accepted that the composition of a meal can be estimated from analysis of the different components of that meal, it is difficult to establish the bioavailability of the meal components. For instance, a change in the plasma concentration of meal components does not relate to its bioavailability. Protein turnover in healthy individuals and during a variety of disease states, including in critically ill patients, has predominantly been calculated using primed-constant and continuous infusion of combinations of stable isotope amino acids such as leucine, phenylalanine and tyrosine.

Protein intake in humans has been studied for many years. The currently used estimated protein intake requirements are based on the requirements established for healthy humans to which a multiplication factor is added. A decreased anabolic response to dietary protein has been observed in older adults. Sarcopenia is the age-related loss of muscle mass and function and affects a significant portion of older adults globally. This condition leads to prolonged hospitalizations, frailty, and financial burdens for both patients and healthcare providers. High protein intake and quality are crucial in preventing and treating sarcopenia by promoting muscle growth. Dietary protein, particularly dairy and plant-based protein, can positively impact these characteristics. However, it remains unclear how consumption of these dietary proteins affects the body's metabolic pathways compared to dairy proteins.

Adequate or optimal protein intake and exercise are of importance to improve muscle health in the older and diseased populations. Still, it remains unclear whether there is anabolic resistance to protein intake. However, the metabolic phenotype of older adults in the prandial state remains unclear as calculation of the anabolic response is hampered by methodological challenges such as amino acid splanchnic extraction measurement and the underestimation of the true amino acid appearance.

Thus, there remain unmet needs in the art for determining amino acid kinetics in relation to the meal composition, particularly in older adults and/or critically ill patients. More particularly, the art is deficient in methods for measuring bioavailability of nutrients and modeling nutrient metabolism after a meal via a combination of compartmental analysis and pulse tracer administration. The present invention fulfills this longstanding need and desire in the art.

### SUMMARY OF THE INVENTION

The present invention is directed to a method for measuring bioavailability of nutrients in a subject after a meal. In this method, the subject is sip-fed over a period of time a meal that contains a mixture of non-labeled nutrients. A pulse of stable isotope-labeled amino acids is administered to the subject during the period of time and a compartmental analysis is performed to model nutrient metabolism in the subject, thereby measuring bioavailability of the nutrients.

The present invention is directed to a related method which further comprises measuring protein quality via quantification of a corresponding metabolic pathway; measuring a response of the metabolic pathway to at least one meal; or estimating an effect on a plurality of amino acid pathways after intake of proteins or specific dietary components; or a combination thereof.

The present invention is further directed to a method for modeling nutrient metabolism in a subject in need thereof. In this method, the subject is fed a meal over a period of time comprising non-labeled amino acids. A pulse tracer of stable isotope-labeled amino acids is administered to the subject during the period of time and a compartmental analysis is performed to measure a response to the meal of at least one metabolic pathway, thereby modeling nutrient metabolism in the subject.

The present invention is directed further to a profile of nutrient metabolism of a subject produced by the method as described herein.

The present invention is directed further still to a method for monitoring nutrient requirements of a patient in need thereof. In this method, a meal is fed to the critically ill patient. Nutrient metabolism in the patient is modeled to produce a profile thereof, where the profile provides bioavailability of the amino acids and an intracellular amino acid response to components in the meal fed to the critically ill patient to determine the nutrient requirements. The present invention is directed to a related method further comprising repeating the feeding step and the modeling nutrient metabolism step periodically to monitor changes in the nutrient requirements of the critically ill patient.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1B compare the prandial metabolic response by analyzing the enrichment of ¹³C-labeled phenylalanine (Phe) in endogenous whole-body protein (FIG. 1A) and in the intracellular disposal of ¹³C-labeled phenylalanine (Phe) (FIG. 1B) after sip-feeding of mixtures of essential amino acids (EAA), complete amino acids (TTA) and/or of a placebo.

FIGS. 2A-2B compare the prandial metabolic response by analyzing the enrichment of ¹³C-labeled phenylalanine (Phe) in endogenous whole body protein (FIG. 2A) and in the intracellular disposal of ¹³C-labeled phenylalanine (Phe) (FIG. 2B) after sip-feeding of mixtures of essential amino acids (EAA), EAA plus hydroxy-methylbutyric acid (EAA+HMB), EAA plus leucine (EAA+LEU) and/or a placebo.

FIG. 3 shows that intracellular phenylalanine production is much higher than whole body production (WBP).

FIGS. 4A-4B show phenylalanine production. WBPfed is the non-compartmental production during feeding. WBPfed-fasted is when the whole body production in the post absorptive condition is subtracted from the whole body production during feeding. Enteral-given is the amount of phenylalanine given with the food. Delta EndoWBP is enteral-given subtracted from WBPfed-fasted, representing the change in the endogenous protein breakdown.

### DETAILED DESCRIPTION OF THE INVENTION

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the terms “comprise” and “comprising” are used in the inclusive, open sense, meaning that additional elements may be included.

As used herein, the terms “consist of” and “consisting of” are used in the exclusive, closed sense, meaning that additional elements may not be included.

As used herein, the term “about” refers to a numeric value, including, for example, whole numbers, fractions, and percentages, whether or not explicitly indicated. The term “about” generally refers to a range of numerical values (e.g., +5-10% of the recited value) that one of ordinary skill in the art would consider equivalent to the recited value (e.g., having the same function or result). In some instances, the term “about” may include numerical values that are rounded to the nearest significant figure.

As used herein, the terms “subject” and “patient” are used interchangeably and refer to a human.

In one embodiment of the present invention, there is provided a method for measuring bioavailability of nutrients in a subject after a meal, comprising sip feeding to the subject over a period of time a meal that contains a mixture of non-labeled nutrients; administering a pulse of stable isotope-labeled amino acids to the subject during the period of time; and performing a compartmental analysis to model nutrient metabolism in the subject, thereby measuring bioavailability of the nutrients.

Further to this embodiment the method comprises measuring protein quality via quantification of a corresponding metabolic pathway; measuring a response of the metabolic pathway to at least one meal; or estimating an effect on a plurality of amino acid pathways after intake of proteins or specific dietary components; or a combination thereof.

In both embodiments, the mixture of non-labeled nutrients may comprise non-labeled amino acids, non-labeled proteins or a combination thereof. Representative stable isotopes include but are not limited to carbon-13, deuterium, nitrogen-15, or a combination thereof. In addition, the period of time for sip feeding may be about 20 minutes for about 6 hours.

In both embodiments, modeling nutrient metabolism in the subject may enable calculating an actual appearance of food-derived amino acids. Also in both embodiments the subject may be a healthy subject or a critically ill subject. Particularly, the critically ill subject may suffer from sarcopenia.

In another embodiment of the present invention, there is provided a method for modeling nutrient metabolism in a subject in need thereof, comprising feeding the subject a meal over a period of time comprising non-labeled amino acids; administering a pulse tracer of stable isotope-labeled amino acids to the subject during the period of time; and performing a compartmental analysis to measure a response to the meal of at least one metabolic pathway, thereby modeling nutrient metabolism in the subject.

In this embodiment, the subject may have a pathophysiological condition associated with a loss of muscle mass or is at risk for the same. A representative example of a pathophysiological condition is sarcopenia. Also in this embodiment, the pulse tracer may comprise amino acids labeled with carbon-13 isotope, deuterium, nitrogen-15, or a combination thereof. The period of time for sip feeding may be about 20 minutes for about 6 hours.

In yet another embodiment of the present invention, there is provided a profile of nutrient metabolism of a subject produced by the method as described supra.

In this embodiment, the measured response may comprise bioavailability of the amino acids in the meal or intracellular amino acid responses to components in the meal or a combination thereof.

In yet another embodiment of the present invention, there is provided a method for monitoring nutrient requirements of a patient in need thereof, comprising feeding a meal to the patient; and modeling nutrient metabolism in the patient to produce a profile thereof, said profile providing bioavailability of the amino acids and an intracellular amino acid response to components in the meal fed to the patient to determine the nutrient requirements. Further to this embodiment, the method comprises repeating the feeding step and the modeling nutrient metabolism step periodically to monitor changes in the nutrient requirements of the patient. In both embodiments the patient may suffer from sarcopenia.

In one aspect of both embodiments of the present invention, the feeding step may comprise sip feeding to the patient over a period of time of about 20 minutes for about 6 hours a meal containing a mixture of non-labeled nutrients comprising non-labeled amino acids, non-labeled proteins or a combination thereof; and administering to the subject during the period of time a pulse of stable isotope-labeled amino acids, where the stable isotope label may comprise carbon-13, deuterium, or nitrogen-15, or a combination thereof.

In another aspect of both embodiments, the modeling nutrient metabolism step may comprise performing a compartmental analysis to measure a response to the meal of one or more metabolic pathways and at least one of: measuring protein quality via quantification of a corresponding metabolic pathway; measuring a response of the metabolic pathway to one or more meals; or estimating an effect on a plurality of amino acid pathways after intake of proteins or specific dietary components.

Provided herein are methods for measuring bioavailability of nutrients and modeling the nutrient metabolism of a subject or patient after ingesting a meal. A combination of compartmental analysis on a mixture of non-labeled nutrients are fed to a subject or patient over a period of time and stable pulse tracer administration of at least one stable isotope-labeled amino acid is utilized which overcomes the deficiencies in the primed-constant and continuous infusion protocols. Moreover, this enables measuring or estimating the effects on amino acid pathways or metabolic pathways after the meal, such as, for example, protein quality by quantifying the corresponding pathway, the response of the metabolic pathway to one or more meals and the effect on amino acid pathways after the intake of proteins or specific dietary components.

The mixture of non-labeled nutrients may comprise non-labeled amino acids or non-labeled proteins or a mixture of both. Examples of stable isotopes are, for example, carbon-13, deuterium, nitrogen-15, or a combination thereof. The subject may be a healthy subject or critically ill. The subject may be an older subject, such as about 60 or older and may be suffering from age-related loss of muscle mass and function or other pathophysiological condition, for example, but not limited to, sarcopenia.

Thus, modeling nutrient metabolism enables monitoring nutritional requirements and devising nutritional approaches for treating patients with sarcopenia or critically ill patients and improving patient outcomes. Upon obtaining an initial nutrient metabolic model of the patient, one of ordinary skill in the art is well-able to calculate the exact nutrient requirements, such as, the exact protein requirements and needs of the specific patient to alleviate or treat the sarcopenia or other pathophysiological condition.

The following examples are given for the purpose of illustrating various embodiments of the invention and are not meant to limit the present invention in any fashion.

### Example 1

Twofold Intake of Free Essential Amino Acids Doubles the Anabolic Response in Healthy Older Adults without Reducing Protein Breakdown

### Method

Using the stable isotope pulse method provided herein, the prandial metabolic response of older adults to free essential amino acids (EAA) mixtures was characterized during sip feeding of two levels of free essential amino acids or essential amino acids as part of a complete amino acids (TAA) mixture using (non)-compartmental analysis that estimates more accurately the whole body anabolic response to meals.

Eleven healthy men and women, aged 60-80, participated in 4 separate study days, >3 days apart, where they consumed by sip feeding every 20 min for 6 hours according to a randomized placebo-controlled single-blind design an oral mixture containing free: a) TAA (45 g, 20 g essential amino acids), b) essential amino acids (20 g), c) EAA-high (45 g), and d) Placebo (water). Three hours after start of intake, a pulse of stable isotope tracers, containing L-[ring-¹³C₆]phenylalanine and L-[ring-²H₄]tyrosine was administered. Blood samples were collected before each sip. Plasma amino acid tracer enrichments were analyzed by LC-MS/MS. (Non)-compartmental (NC or C) analysis to estimate increase (delta) above placebo of WbProteinSynthesis (dWbPS), WbEndogenous Protein breakdown (dWbEndoPB) and netWbPS (net anabolism) from isotope decays calculations and statistical analysis (ANOVA) by Graphpad Prism. Significance: P<0.05. Results are expressed as mean [95% Cl].

### Results

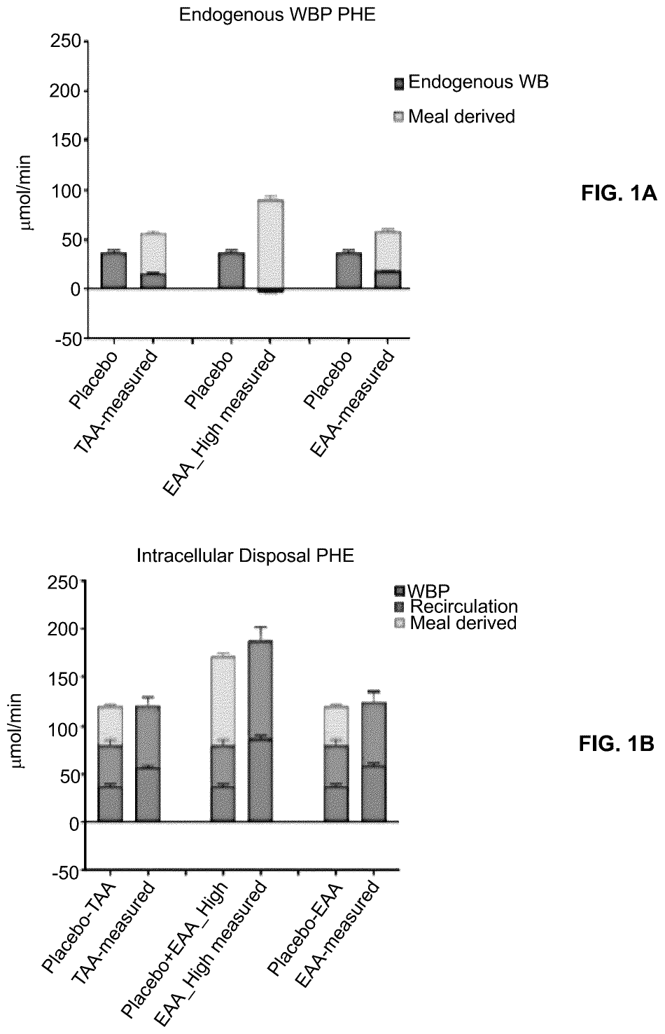

In FIGS. 1A-1B, essential amino acids vs complete amino acids did not show any differences in measured NC (non-compartmental) or C (compartmental) analysis parameters. EAA-high (Table 1) showed an increase in NetWbPS, related to the intake, using both NC and C calculations (p<0.0001, p=0.0006). dWbEndoPB was increased after essential amino acids-high intake using C analysis but decreased using NC analysis. Values are estimated mean [95% Cl], expressed in umol/min above placebo group. Intake PHE=40.4 μmol/min. Essential amino acids high intake PHE=90.9 μmol/min.

TABLE 1

| | NC | NC | NC |
|---|---|---|---|
| Mixture | dWbPS | dWbEndoPB | NetWbPS |
| TAA | 17.4 | −21.3 | 38.7 |
| | [14.5, 20.3] | [−23.0, −19.6] | [35.3, 42.1] |
| EAA | 18.6 | −19.2 | 37.8 |
| | [15,6, 21.5] | [−20.9, −17.5] | [34.4, 41.2] |
| | #=0.567 | #=0.139 | #=0.721 |
| EAA-high | 42.7 | −41.4 | 84.1 |
| | [38.7, 46.8] | [45.2, −37.5] | [78.6, 89.7] |
| | #=<0.0001 | #=<0.0001 | #=<0.0001 |
| | C | C | C |
| Mixture | dWbPS | dWbEndoPB | NetWbPS |
| TAA | 37.7 | 1.2 | 36.5 |
| | [25.1, 50.3] | [−6.4, 8.9] | [21.7, 51.2] |
| EAA | 39.7 | 5.3 | 34.4 |
| | [26.9, 52.5] | [−2.3, 13.0] | [19.5, 49.3] |
| | #=0.828 | #=0.488 | #=0.849 |
| EAA-high | 91.9 | 16.5 | 75.4 |
| | [73.9, 109.8] | [6.0, 26.9] | [54.6, 96.2] |
| | #=<0.0001 | #=0.011 | #=0.0006 |
| | | *p=0.061 | *=0.0003 |

#=p vs TAA,
*=p vs EAA

The presence of non-essential amino acids in a TAA mixture does not affect the anabolic response. Doubling the amount of essential amino acids doubles the anabolic response in older adults, independent of the (non-) compartmental model used. However, the underlying mechanism of the anabolic response of essential amino acids-high, using the compartmental model, indicates an overall increase of protein breakdown and synthesis. It is concluded that compartmental modeling matches the true physiological metabolism of net protein synthesis more accurately than non-compartmental analysis.

### Example 2

Non- or Compartmental Analysis of Meal Anabolic Capacity after Pulse Tracer Administration

### Method

Similarly to Example 1, the stable isotope pulse method is used to characterize the prandial metabolic response of older adults to free essential amino acids mixtures during sip feeding of free essential amino acids, essential amino acids plus hydroxy-methylbutyric acid (EAA+HMB) or essential amino acids plus leucine (EAA+LEU using (non)-compartmental analysis that estimates more accurately the whole body anabolic response to meals.

Eleven healthy men and women, aged 60-80 years, participated in 4 separate study days, >3 days apart, where they consumed by sip feeding every 20 min for 6 hours according to a randomized placebo-controlled single-blind design an oral mixture containing: a) essential amino acids (20 g), b) EAA+HMB (20+3 g), c) EAA+LEU (20+3 g), d) and Placebo (water). Three hours after start of intake, a pulse of stable isotope tracers, containing L-[ring-¹³C₆]phenylalanine and L-[ring-²H₄]tyrosine was administered. Blood samples were collected before each sip. Plasma amino acid tracer enrichments were analyzed by LC-MS/MS. (Non)-compartmental (NC or C) analysis to estimate increase (delta) above placebo of WbProteinSynthesis (dWbPS), WbEndogenous Protein breakdown (dWbEndoPB) and netWbPS (net anabolism) from isotope decays calculations and statistical analysis (ANOVA) by Graphpad Prism. Significance: P<0.05. Results are expressed as mean [95% CI].

### Results

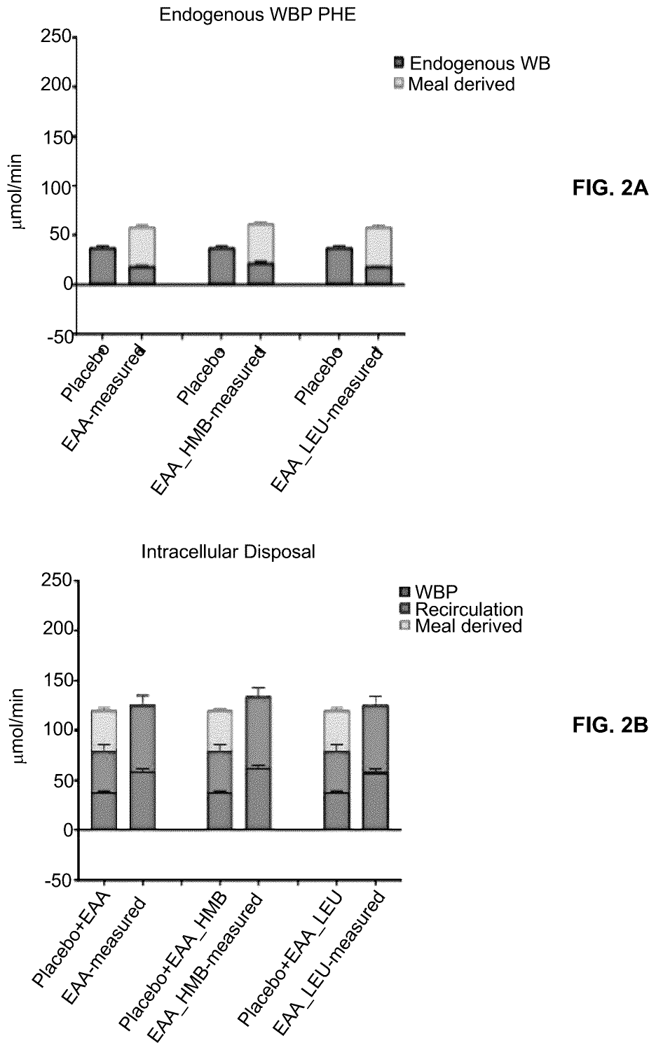

No differences (Table 2) were observed in dWbPS, dWbEndoPB, or netWbPS after EAA+HMB, measured by NC or C analysis (FIGS. 2A-2B). Using NC or C analysis, all mixtures had >80% efficiency in converting intake. Only NC analysis showed that EAA+HMB stimulated dWbPS (p=0.021) and dWbEndoPB (p=0.006) more than EAA+LEU, but not NetWbPS. Values are estimated mean [95% Cl], expressed in μmol/min above placebo group. Intake PHE=40.4 μmol/min.

TABLE 2

| Mixture | Non-Compartmental | | |
|---|---|---|---|
| EAA | 18.6 | −19.2 | 37.8 |
| | [15.6, 21.5] | [−20.9, −17.5] | [34.4. 41.2] |
| EAA + | 22.1 | −15.6 | 37.7 |
| HMB | [19.0, 25.2] | [−17.3, −13.9] | [34.1, 41.2] |
| | #=0.094 | #=0.011 | #=0.965 |
| EAA + | 17.2 | −19.5 | 36.7 |
| LEU | [14.3, 20.1] | [−21.2, −17.8] | [33.3, 40.1] |
| | #=0.517 | #=0.833 | #=0.675 |
| | *=0.021 | *=0.006 | *=0.708 |
| Mixture | Compartmental | | |
| TAA | 39.7 | 5.3 | 34.4 |
| | [26.9, 52.5] | [−2.3, 13.0] | [19.5, 49.3] |
| EAA | 47.3 | 13.0 | 34.2 |
| | [33.9, 60.6] | [5.4, 20.7] | [18.9, 49.6] |
| | #=0.413 | #=0.189 | #=0.988 |
| EAA-high | 36.6 | 4.6 | 32.0 |
| | [23.8, 49.4] | [−3.9, 12.3] | [17.1, 46.0] |
| | #=0.735 | #=0.905 | #=0.825 |
| | *=0.248 | *=0.152 | *=0.837 |

The results, using (non) compartmental analysis after pulse tracer administration, do not support a higher anabolic capacity (NetWbPS) in older adults when adding HMB or LEU to an essential amino acids mixture. Using the compartmental model, essential amino acid mixtures do not reduce endogenous protein breakdown. The present pulse isotope analysis described herein provides new insights in the underlying mechanisms of the anabolic capacity of meals.

### Example 3

### Compartmental Analysis to Establish Actual Protein Breakdown and Dietary Requirements in the Critically Ill

### Protein Turnover Measurement in the Post Absorptive State

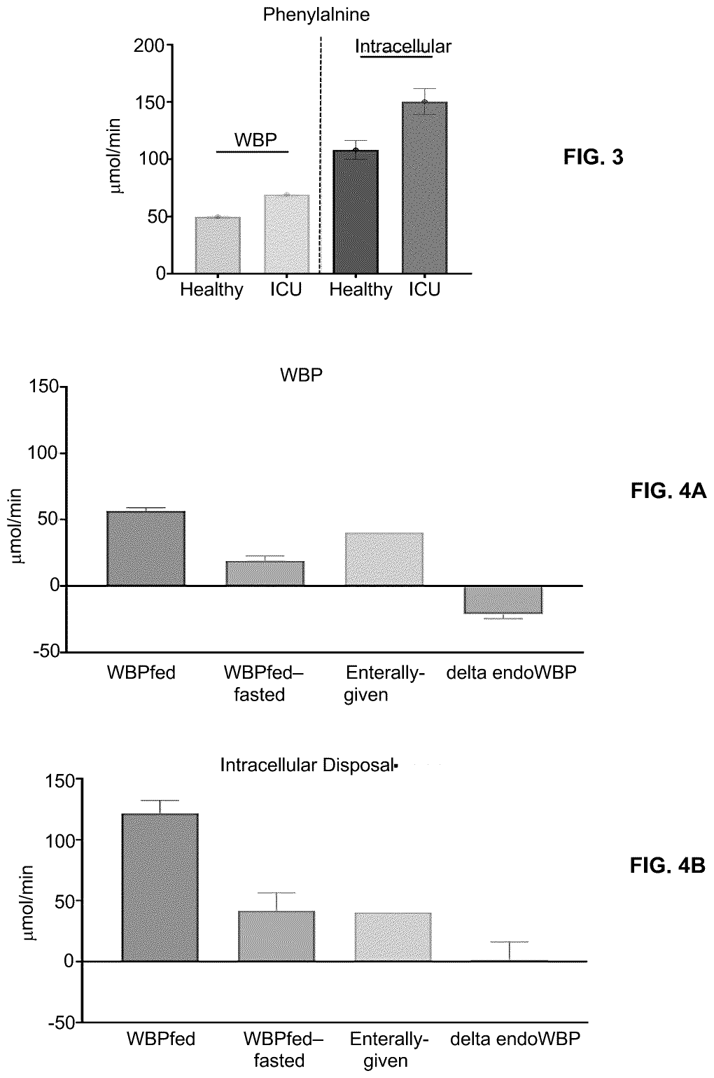

By measuring the phenylalanine production in healthy and critically ill patients (FIG. 3), the turnover of protein per gram protein/day/subject may be calculated (Table 3). As previously reported, about 300 grams of protein/day is broken down in a healthy individual in the postabsorptive state when measured with the primed-constant and continuous stable isotope infusion protocol. If food only increases protein synthesis, dietary intake of about 75 grams of protein/day would increase protein synthesis to 375 grams, increasing protein synthesis by about 25%.

However, calculating protein breakdown from intracellular production (Table 3) leads to about 650 grams/day of protein breakdown in the postabsorptive state. In this case, dietary intake of about 75 grams protein/day would only lead to an 11% increase in protein synthesis. Consequently, if the net protein loss is calculated, which is the difference between protein breakdown and synthesis, a net loss of 87 grams protein/day takes place in a healthy individual when there is no food intake.

In critically ill patients (Table 3), both non-compartmental protein breakdown (PB) and intracellular protein breakdown are increased by about 40% as compared to the healthy state. When using the intracellular protein breakdown measurement, protein degradation is about 900 grams/day and net protein loss is about 60 grams/day which clearly shows that the turnover of protein is substantially increased in relation to the net loss. However, the net loss in critically ill patients is only 6.6% of total protein breakdown. One remarkable observation is that net protein breakdown in the critically ill patients is not increased but decreased in the post absorptive state (Table 3). According to the calculations in relation to loss of lean mass (Table 4), critically ill patients will still lose about 400 grams of lean tissue/day (0.8%/day when total lean mass is about 50 kg). Others have found that loss of muscle mass, which is about 50% of total lean mass in critically ill patients in the ICU is about 1%/day.

So the question arises how loss of body protein be attenuated in healthy subjects in the postabsorptive state? In Table 3 it was calculated that net protein loss in healthy subjects is about 87 grams, indicating that at least 87 grams of dietary amino acids is needed for a healthy subject to become anabolic. When protein is ingested, other factors like digestion play a role leading to a reduced protein efficiency which may partly explain the higher protein intake advised. Reduced digestibility of dietary proteins probably becomes even more important in critically ill patients.

It is observed that an increase in the amount of protein intake in critically ill patients is not needed as much as that the upregulation in protein breakdown needs to be reduced. It is hypothesized that critically ill patients need dietary components that are able to reduce protein breakdown. A reduction in protein breakdown when providing HMB to critically ill patients was recently shown. Moreover, it is contemplated that, as with arginine in the critically ill, other dietary amino acids and/or proteins may also reduce protein breakdown.

TABLE 3
Recalculation of postabsorptive protein
breakdown (PB) rates in humans

| | | | ICU minus |
|---|---|---|---|
| | Healthy | ICU | healthy |
| Non-compartmental | 291.7 | 411.0 | 115.2 |
| PB (WBP) | [286.3, 304.7] | [398.0, 423.5] | [99.8, 131.3] |
| Non-compartmental | 44.0 | 30.7 | −13.4 |
| Net PB | [41.5, 46.6] | [28.5, 32.8] | [−16.8, −10.0] |
| Intracellular PB | 641.5 | 892.2 | 250.7 |
| | [592.2, 690.8] | [824.5, 959.9] | [166.9, 334.4] |
| Intracellular | 86.6 | 60.3 | −26.3 |
| Net PB | [81.5, 91.6] | [55.9, 64.6] | [−32.9, −19.6] |

Data are grams of protein/day (mean [95% CI]) and obtained from recalculation of data. Phenylalanine and tyrosine decay curve parameters were used to calculate the non-compartmental PB (comparable to rate of appearance in plasma), intracellular production and net protein breakdown, using the conversion of phenylalanine to tyrosine. It was assumed that 4% of protein is phenylalanine.

TABLE 4
Net lean mass loss in humans when no food is provided

| Healthy | 577 gram |
|---|---|
| | [544, 611] |
| Critically III | 402 gram |
| | [373, 431] |
| ICU minus healthy | −175 gram |
| | [−220, −131] |

Data are gram lean mass/day loss, calculated from intracellular net PB, assuming lean mass contains 15% protein.

### Protein Breakdown and Synthesis During Feeding

During feeding, the amino acids released into the circulation will increase due to enhanced digestion and absorption of the meal-derived amino acids and these amino acids becoming available from intracellular protein breakdown. The increased appearance of amino acids into the circulation and intracellularly will stimulate the disposal of amino acids (mainly for protein synthesis), and an increased intracellular concentration could reduce protein breakdown. Several studies have observed a reduced endogenous protein breakdown when using the primed-constant and continuous tracer infusion model.

However, it remained unclear whether proteins in the meal are indeed able to reduce protein breakdown. A study in eleven human subjects was performed recently to examine whether intracellular protein breakdown (using compartmental analysis) also may be measured in the prandial state. For that purpose, a protocol was developed in which nutrition was provided every 20 min as sips containing a mixture of free amino acids, representing the composition of whey protein. After steady state was obtained, as verified by adding stable isotopes of amino acids to the sips and measuring the plasma enrichment and concentration of these amino acids, the pulse of stable isotopes was administered as previously done in the postabsorptive condition.

The turnover of phenylalanine (as a measure of protein breakdown) obtained by non-compartmental was subsequently compared with intracellular (compartmental) analysis in the prandial state (FIG. 4A). Non-compartmental protein breakdown (WBP), measured during feeding (WBPfed), was about 50 μmol/min using this approach. When subtracting the WBP when no food was given (WBPfed-fasted), the difference in WBP was about 20 μmol/min. As the amount of phenylalanine given enterally was larger than the increase of the WBPfed-fasted, the difference became negative (delta endoWBP). This means that there is a reduction in endogenous protein breakdown (endoWBP) when calculating the effect of feeding. A consistent reduction of protein breakdown during feeding has previously been observed by us and others when WBP was measured using the primed-constant and continuous infusion model.

However, as indicated above, the non-compartmental protein breakdown calculation underestimates the true intracellular protein breakdown, suggesting that the method of calculation of the intracellular protein breakdown provided herein provides a better reflection of the true protein breakdown. When the same calculations were done using the intracellular protein breakdown approach (FIG. 4B), the amount of phenylalanine, given enterally as sips, was very well matched with the difference between the intracellular appearance fed and fasted. Therefore, no reduction in protein breakdown was observed anymore. It is therefore believed that feeding does not reduce endogenous protein breakdown.

What is claimed is:

1. A method for measuring bioavailability of nutrients in a subject after a meal, comprising:
sip feeding to the subject over a period of time a meal that contains a mixture of non-labeled nutrients;
administering a pulse of stable isotope-labeled amino acids to the subject during the period of time; and
performing a compartmental analysis to model nutrient metabolism in the subject, thereby measuring bioavailability of the nutrients.

2. The method of claim 1, wherein the mixture of non-labeled nutrients comprises non-labeled amino acids, non-labeled proteins or a combination thereof.

3. The method of claim 1, wherein the stable isotope is carbon-13, deuterium, nitrogen-15, or a combination thereof.

4. The method of claim 1, wherein the period of time for sip feeding is about 20 minutes for about 6 hours.

5. The method of claim 1, wherein modeling nutrient metabolism in the subject enables calculating an actual appearance of food-derived amino acids.

6. The method of claim 1, further comprising:
measuring protein quality via quantification of a corresponding metabolic pathway;
measuring a response of the metabolic pathway to at least one meal; or
estimating an effect on a plurality of amino acid pathways after intake of proteins or specific dietary components;
or a combination thereof.

7. The method of claim 1, wherein the subject is a healthy subject or a patient.

8. The method of claim 7, wherein the patient subject suffers from sarcopenia.

9. A method for modeling nutrient metabolism in a subject in need thereof, comprising:
feeding the subject a meal over a period of time comprising non-labeled amino acids;
administering a pulse tracer of stable isotope-labeled amino acids to the subject during the period of time; and
performing a compartmental analysis to measure a response to the meal of at least one metabolic pathway, thereby modeling nutrient metabolism in the subject.

10. The method of claim 9, wherein the subject has a pathophysiological condition associated with a loss of muscle mass or is at risk for the same.

11. The method of claim 10, wherein the pathophysiological condition is sarcopenia.

12. The method of claim 9, wherein the pulse tracer comprises amino acids labeled with carbon-13 isotope, deuterium, nitrogen-15, or a combination thereof.

13. The method of claim 9, wherein the period of time for sip feeding is about 20 minutes for about 6 hours.

14. A profile of nutrient metabolism of a subject produced by the method of claim 9.

15. The profile of claim 14, wherein the measured response comprises bioavailability of the amino acids in the meal or intracellular amino acid responses to components in the meal or a combination thereof.

16. A method for monitoring nutrient requirements of a patient in need thereof, comprising:
feeding a meal to the patient; and
modeling nutrient metabolism in the patient to produce a profile thereof, said profile providing bioavailability of the amino acids and an intracellular amino acid response to components in the meal fed to the patient to determine the nutrient requirements.

17. The method of claim 16, further comprising repeating the feeding step and the modeling nutrient metabolism step periodically to monitor changes in the nutrient requirements of the patient.

18. The method of claim 16, wherein the feeding step comprises:
sip feeding to the patient over a period of time of about 20 minutes for about 6 hours a meal containing a mixture of non-labeled nutrients comprising non-labeled amino acids, non-labeled proteins or a combination thereof; and
administering to the subject during the period of time a pulse of stable isotope-labeled amino acids, said stable isotope label comprising carbon-13, deuterium, or nitrogen-15, or a combination thereof.

19. The method of claim 16, wherein the modeling nutrient metabolism step comprises:
performing a compartmental analysis to measure a response to the meal of one or more metabolic pathways and at least one of:
measuring protein quality via quantification of a corresponding metabolic pathway;
measuring a response of the metabolic pathway to one or more meals; or
estimating an effect on a plurality of amino acid pathways after intake of proteins or specific dietary components.

20. The method of claim 16, wherein the critically ill patient suffers from sarcopenia.
